(19) United States
(12) Patent Application Publication
(10) Pub. No.: US 2006/0079416 A1
(43) Pub. Date: Apr. 13, 2006

(54) SYSTEM AND METHOD FOR A MESH CLEANSING DEVICE

(75) Inventor: E. Gary Myers, Scottsdale, AZ (US)

Correspondence Address: SNELL & WILMER, LLP, ONE ARIZONA CENTER, 400 E. VAN BUREN, PHOENIZ, AZ, 85004-2202, US

(73) Assignee: THE DIAL CORPORATION, Scottsdale, AZ (US)

(21) Appl. No.: 11/162,933

(22) Filed: Sep. 28, 2005

Related U.S. Application Data
(60) Provisional application No. 60/684,841, filed on May 25, 2005.
(60) Provisional application No. 60/701,669, filed on Jul. 21, 2005.
(60) Provisional application No. 60/613,884, filed on Sep. 28, 2004.

(51) Int. Cl. A61K 8/00

(52) U.S. Cl. 510/130

(57) ABSTRACT

The present invention comprises a mesh cleansing device, generally known as a pouf. The pouf comprises a mesh body that encases a tulle pouch, wherein the tulle pouch further encases pelletized soap. The pouf also comprises a lanyard that acts as a means for securing the shape of the pouf, maintaining the tulle pouch within the pouf, facilitate holding the pouf by a user, and providing a means to hang the pouf during non-use. The pouf also comprises a secondary string to maintain the shape of the pouf. In accordance with an exemplary embodiment of the invention, the shape of the pouf comprises a round elongated body about a horizontal plane, having a generally flat upper and lower region, or, 37 pumpkin” shape.

### CROSS-REFERENCE TO RELATED APPLICATIONS

This application claims priority to U.S. Provisional Patent Application, Ser. No. 60/701,669 entitled “System and Method for a Mesh Cleansing Device” filed on Jul. 21, 2005, and U.S. Provisional Patent Application Ser. No. 60/613,884 entitled “Mesh Bathing Device” filed on Sep. 28, 2004, and U.S. Provisional Application Ser. No. 60/684,841 entitled “Mesh Bathing Device” filed on May 24, 2005.

### FIELD OF INVENTION

This invention relates to a system and method for a mesh cleansing device, commonly referred to as a pouf, and more specifically, to a pouf that encases an internal tulle pouch that in turn encases a soap cleansing product.

### BACKGROUND OF INVENTION

For centuries, people have used washcloths and soaps for cleaning their body to effectuate basic, general hygiene. Traditionally, a soap bar was used with a washcloth, wherein a person laboriously massaged the soap bar with the washcloth to generate soap lather. The soap lather was then applied to the body with the washcloth and rubbed about with the washcloth. The washcloth was used in this fashion to distribute the soap about the body, providing a mild skin exfoliation and upon rinsing the soap from the body, body cleaning was complete.

With the introduction of liquid soaps, soap lather became easier to generate, but a typical washcloth still tended to “push” the lather around instead of working the lather in the desired area. Moreover, a typical washcloth was often difficult to manipulate. The washcloth often had to be “bunched up” to provide proper holding for a proper cleansing.

With the advent of synthetic polymers, many newer cleansing devices have been developed. For example, most recently, gathered synthetic mesh cleansing devices, commonly known in the art as “poufs” have been developed. They are popular because they are easy to manipulate, are efficient in generating soap lather, exfoliate in a superior manner, and effectuate an easy way to distribute lathered soap about the body. In other words, poufs are advantageous over a typical washcloth that is often difficult to handle, tends to “push” around a generated soap lather, and then, only after a person expends significant effort generating lather from the soap by excessive massaging of the soap bar with the washcloth.

With the introduction of liquid soaps, used in combination with a mesh cleansing device such as a pouf, personal hygiene has evolved. While the pouf and liquid soap combination effectuates a more efficient cleansing device, the combination still requires the operation of a pouf and a separate soap product. In an effort to provide an even more consumer friendly product, there is a need for a mesh cleansing device, a pouf, that comprises a soap product within it. This allows a user to manipulate only one product. Moreover, to effectuate an even greater cleaning by the device, what is needed is a pouf that has an elongated body with a generally flat upper and lower region, i.e., “pumpkin” shaped. This shape provides a greater surface area for cleaning, as opposed to a mere tangential pouf-body relationship with a traditionally, “round” pouf.

### SUMMARY OF INVENTION

As set forth in the detailed description and the accompanying figures, the present invention includes, in various exemplary embodiments, a mesh cleansing device with an internal, encased soap product. In accordance with an exemplary embodiment, the mesh cleansing device comprises a synthetic mesh, gathered and bound to form what is commonly referred to as a pouf. The pouf comprises an inner fine mesh pouch (tulle pouch), which further encases the soap product. The soap product comprises generally a soluble cleansing composition such as a fatty acid soap, synthetic detergent, combination soap/synthetic, or solid syndet. In a preferred embodiment, the combination of soap and synthetic components are present in a range of about 80:20 to about 20:80 fatty acid soap to synthetic. More preferably, the combination of soap and synthetic components are present in a range of about 40:20 to about 20:40 fatty acid soap to synthetic.

In accordance with an exemplary embodiment, it has been found that by enclosing pieces of the soap product within a tulle pouch and then placing this within the interior of the pouf, the life of the soap is extended. The tulle pouch, a fine mesh bag, is held within the pouf by a lanyard, and the lanyard also acts as a means to handle the pouf, as well as a means for hanging the pouf during non-use. The lanyard is further configured to bind the mesh into the gathered, or pouf shape. In accordance with an exemplary embodiment of the invention, the pouf shape instead of being traditionally round, is configured to comprise a round elongated body with a generally flat upper and lower region, similar to a, “pumpkin” shape. A secondary string opposite the lanyard is also used to maintain the gathering of the synthetic mesh into the, “pumpkin” shape.

Further, in accordance with and exemplary embodiment of the present invention, a personal cleansing device comprises, a gathered mesh body having an elongated body about a horizontal plane, a tulle pouch configured to reside within the interior of the mesh body, a pelletized soap encased by the tulle pouch, a lanyard configured to affix the tulle pouch within the interior of the mesh body and secure the gathering of the mesh body, and a secondary string configured to secure the gathering of the mesh body at its lower region.

In accordance with an exemplary embodiment of the present invention, a method for producing a mesh cleansing device comprises, cutting a length of tubular mesh, placing substantially parallel rods through the tubular mesh, placing a soap filled tulle pouch within the tubular mesh, gathering the tubular mesh, securing the gathering of the tubular mesh and the soap filled tulle pouch within the tubular mesh with a lanyard, securing the gathering of the tubular mesh on the opposite side of the lanyard with a secondary string, and removing the rods.

### BRIEF DESCRIPTION OF THE DRAWINGS

Additional embodiments of the invention will become evident upon reviewing the non-limiting embodiments described in the specification and the claims, in conjunction with the accompanying figures, wherein:

FIG. 1 is an illustration of an exemplary embodiment of a mesh cleansing device showing the general overall shape, an elongated device with a generally flat upper and lower region;

FIG. 2 is an illustration of an exemplary embodiment of a mesh cleansing device showing a region of the pouf removed to illustrate the inner tulle pouch encasing the pelletized soap, attached lanyard, and secondary string;

FIG. 3 is an illustration of an exemplary embodiment showing one aspect of forming the pouf;

FIG. 4 is an illustration of an exemplary embodiment showing the netting used to form the mesh cleansing device body, pouf;

FIG. 5 is an illustration of an exemplary embodiment showing the positioning of the soap encasing tulle pouch in relation to the pouf mesh body;

FIG. 6 is an illustration of an exemplary embodiment showing the positioning of the lanyard about the pouf and internal tulle pouch;

FIG. 7 is an illustration of an exemplary embodiment showing the lanyard secured to the pouf; and

FIG. 8 is an illustration of an exemplary embodiment showing the secondary string securing the bottom region of the pouf.

### DETAILED DESCRIPTION

The detailed description of various exemplary embodiments of the invention herein makes reference to the accompanying figures. While these exemplary embodiments are described in sufficient detail to enable those skilled in the art to practice the invention, it should be understood that other embodiments may be realized and that logical and mechanical changes may be made without departing from the spirit and scope of the invention. Thus, the detailed description herein is presented for purposes of illustration only and not of limitation. In accordance with an exemplary embodiment, other configurations may be used. For example, it may be determined that a mesh cleansing device having multiple tulle pouches is beneficial, or tulle pouches having various sizes and shapes even further extend a soap life.

In various exemplary embodiments of the invention, a pouf is described as a personal cleansing device for effectuating personal hygiene. It should be noted, however, that the pouf may be suitably configured to be used as a cleaning device for any number of applications, for example, household use, appliances, vehicles, pets, outdoor use, and any application wherein cleaning procedures are conducted.

For the sake of brevity, the components of the device which may be known to one skilled in the art may not necessarily be described in great detail herein. Furthermore, the connecting lines illustrated in the various figures contained herein are intended to represent exemplary functional relationships and/or physical couplings between the various elements. It should be noted that many alternative or additional functional relationships or physical connections may be present in a practical system or method.

In accordance with an exemplary embodiment of the invention, a pouf is used as a personal cleansing device. A user handles the pouf by grasping the body of the pouf or, alternatively, by it's lanyard. Upon bathing, the user immerses the pouf amid water, wherein the water flows through body mesh openings and interacts with an inner tulle pouch. The inner tulle pouch, which encases pelletized soap, is configured with mesh openings to allow the water to interact with the encased pelletized soap. The pelletized soap is configured to be water soluble, dissolving upon contact with the water. The interaction among the foregoing elements allows the user to generate a soap lather that the user may work throughout their body, thereby effectuating cleansing of the user's body.

In accordance with various embodiments of the present invention, elements of the pouf may vary depending on the needs and tastes of the user. For example, the pouf may be configured to accommodate different users, i.e., children may require a smaller pouf, while adults may desire a larger pouf. In another example, the pouf may comprise various colors to accommodate the various decorative tastes among consumers. Additionally, the pouf, comprising pelletized soap, may comprise various characteristics, such as, variable fragrances, skin conditioners, and the like.

That said, various exemplary embodiments of the present invention, a personal cleansing device, referred to herein as a pouf, are considered. The pouf comprises, in general, a pouf body, a tulle pouch, and soap. The pouf comprises a generally synthetic mesh material and is used to facilitate working up of soap lather, as well as, distribute the soap lather about the body of a user. It should be noted that natural or organic materials may also be used to formulate the pouf. The synthetic mesh material is gathered into a roughly round, “pumpkin,” shape, thereby creating the pouf body, but other configurations may be used, such as an elongated shape. Moreover, the pouf may comprise various geometric configurations, like a torus shape. In its gathered state, the pouf body is easily held by a user, allowing a user during bathing to execute cleaning and a superior exfoliation.

As noted above, in various embodiments, the pouf includes an inner tulle pouch. The tulle pouch comprises a generally fine mesh material, wherein the material is configured into a pouch form to encase a soap composition. The tulle pouch comprises mesh openings that are large enough to allow water to readily pass through, but small enough to prevent the encased soap from escaping. Described in greater detail below, the tulle pouch may comprise of various configurations and material. In one example, the pouf comprises a single soap encasing tulle pouch, however, it should be noted that multiple tulle pouches may be used, or a single tulle pouch having multiple compartments.

As noted above, in various embodiments, the pelletized soap comprises a generally water soluble composition. The soap is configured to maintain a solid form such that it readily resides within the tulle pouch, however, it is soluble to the extent that upon use of the pouf device, an appropriate amount of soap dissolves in the water and allows a user to generate soap lather. In one example, the soap is described in a pelletized form, however, it should be realized that any type of soap composition that may be readily contained within a tulle pouch, yet dissolve within water to the extent that the pouf device can be properly used, may be considered. For example, soap paste, soap chips, bars, and the like may be used.

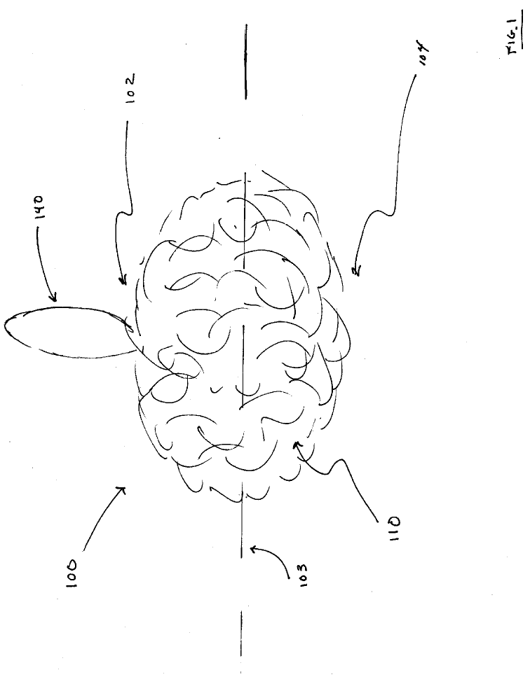

With reference to FIG. 1, in an exemplary embodiment of the invention, pouf 100 comprises a synthetic mesh material, configured in a generally, round elongated body 110, about a horizontal plane 103. The mesh body 110 has generally flat upper and lower regions 102 and 104, and in this exemplary embodiment, mesh body 110 comprises a, “pumpkin” shape.

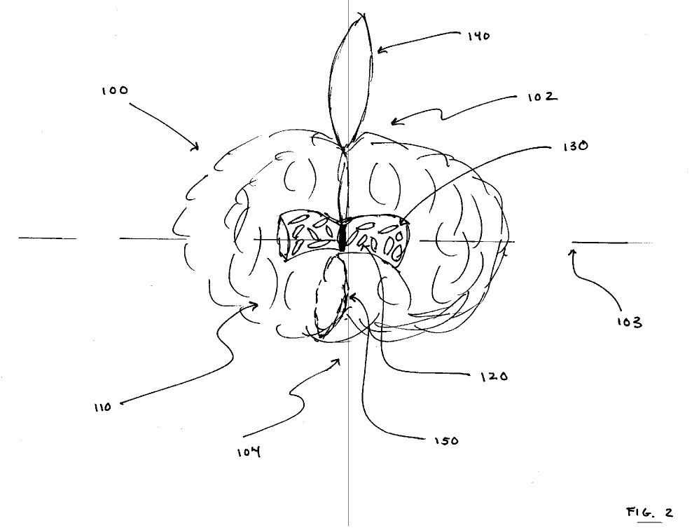

As noted above, pouf 100 is configured to enclose a tulle pouch 130. For example, with reference to FIG. 2, pouf encloses a finer internal mesh bag (tulle pouch) 130, and which in turn is configured to encase pelletized soap 120. In accordance with various embodiments, tulle pouch 130 is secured within pouf body 110 by a lanyard 140. In accordance with various aspects of the present invention, lanyard 140 is further configured to act as a means to manipulate pouf 100 by a user, and facilitate storage by hanging when pouf 100 is not in use. Moreover, lanyard 140, along with a secondary string 150, may be configured to bind the gathering of mesh body 110 and secure the shape of pouf 100 into the round, elongated shape, having flat upper and lower regions 102 and 104, as described above and in greater detail below.

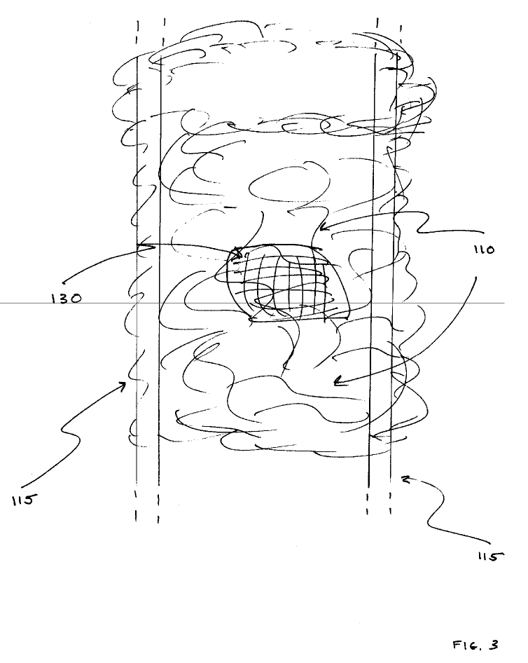

In accordance with an exemplary embodiment of the invention, mesh body 110 is formed from an elongated tubular netting mesh such as illustrated in FIG. 3. In one example, mesh body 110 may be prepared from a flexible polymer comprising, addition polymers of olefin monomers and polyamides of polycarboxylic acids and polyamines. In another example, mesh body 110 may be prepared from LDPE, EVA, and colorants. However, it should be noted that other combinations of synthetic materials not now known or developed in the future may likewise be used.

In accordance with an exemplary embodiment of the invention and with continued reference to FIG. 3, mesh body 110 that may be used to produce the approximately 40 gram adult pouf, is about 4 meters long, having a width of about 40 centimeters. Alternatively, mesh body 110 that may be used to produce the approximately 35 gram children's pouf, is about 2.8 meters long, also having a width of about 40 centimeters. In general, pouf 100 may comprise of any desired size, defined by weight wherein about 10-15 grams per meter of mesh body 110, having a width of approximately 40 centimeters.

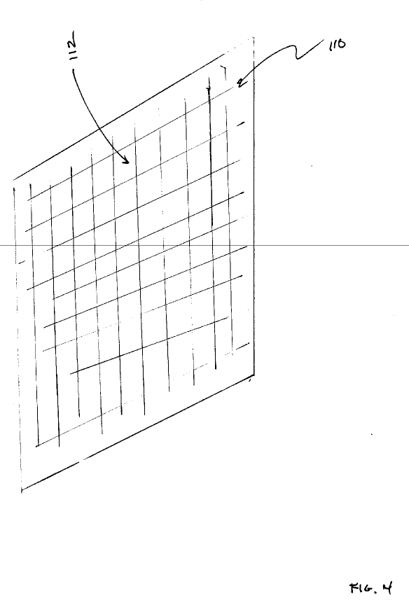

In accordance with an exemplary embodiment of the invention and illustrated by FIG. 4, mesh body 110 comprises mesh openings 112 comprising about 1 to about 65 mesh openings per square centimeter, preferably about 20 to about 45 mesh opening per square centimeter. It should be noted that other configurations of mesh body 110 may be used. For example, a greater or lower density of openings per square centimeter may be used. Also, instead of having uniform size openings, openings 112 may comprise random sizes. In addition, openings 112 may comprise regular configurations such as square, rectangular, triangular, hexagonal, and the like, or openings 112 may comprise irregular configurations too.

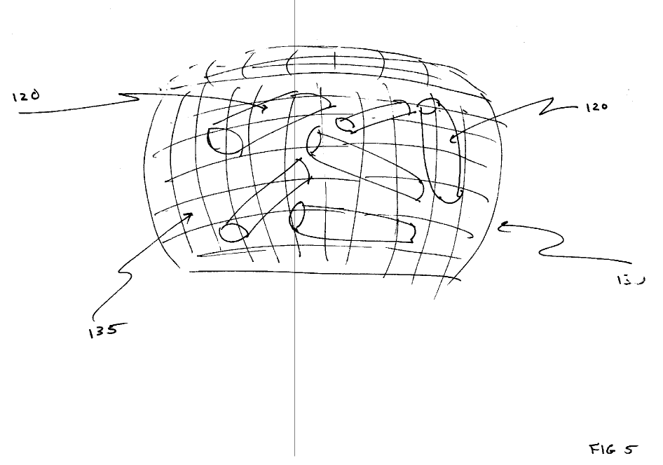

In accordance with an exemplary embodiment of the invention, generally illustrated in FIG. 5, pouf 100 comprises inner tulle pouch 130. As noted above, tulle pouch 130 is configured to act as a pouch to encase pelletized soap 120. In this exemplary embodiment, tulle pouch 130 is sealed about the perimeter to prevent pelletized soap 120 from falling out of tulle pouch 130. In accordance with one aspect of the invention, tulle pouch 130 is substantially hidden from view by gathered mesh body 110.

In accordance with the presently described exemplary embodiment, tulle pouch 130 comprises tulle pouch mesh openings 135. Tulle pouch openings 135 comprise openings small enough to prevent pelletized soap 120 from falling out, but large enough to allow water to easily reach pelletized soap 120 and readily dissolve it. In this embodiment, openings 135 comprise about 60 to about 120 openings per square centimeter. Similar to mesh body 110, it should be noted that other configurations of openings 135 may be used. For example, a greater or lower density of openings per square centimeters may be used. Also, instead of having uniform size openings, openings 135 may comprise random sizes. In addition, openings 135 may comprise regular configurations such as square, rectangular, triangular, hexagonal, and the like, or openings 135 may comprise irregular configurations too. In accordance with an aspect of the invention, the tulle-pouf opening ratio comprises the ratio of the number of openings per square centimeter in the tulle pouch to the number of openings per square centimeter in the pouf. In accordance with a preferred embodiment, the tulle-pouf opening ratio is about 50:1 to about 1:1. In a more preferred embodiment, the tulle-pouf opening ratio is about 4:1 to about 1:1. In a most preferred embodiment, the tulle-pouf opening ration is about 3:1.

In accordance with the present descriptive embodiment of the invention, pelletized soap 120 is of a high water content soap formula. As used herein the term “soap” comprises a water soluble cleansing composition for personal use such as a fatty acid soap or a synthetic detergent or a combination of such a fatty acid soap and synthetic detergent, or solid syndet. In a preferred embodiment, the combination of soap and synthetic components are present in a range of 80:20 to 20:80 fatty acid soap to synthetic. More preferably, the combination of soap and synthetic components are present in a range of 40:20 to 20:40 fatty acid soap to synthetic. As such, water content in the soap is higher than “conventional” soaps. Soap 120 is manufactured by any conventional means now known or as yet known.

For example, approximately 10 to about 90 grams of pelletized soap is encased by tulle pouch 130. In a more preferred example, about 40 to about 60 grams of pelletized soap is used. Soap pellet composition comprises about 2% to about 25% by weight of moisture. In another preferred example, soap pellet composition comprises about 8% to about 25% by weight of moisture. In a most preferred example, soap pellet composition comprises about 10% to about 22% by weight.

The amount of soap in the pellets comprises about 50% to about 100% by weight. In another preferred example, the amount of soap in the pellets comprises about 84% to about 95% by weight. In a most preferred example, the amount of soap in the pellets comprises about 90% to about 95%. Pelletized soap 120 may be configured to comprise about 86% by weight of sodium soap, that is, sodium salt of fatty acids such as sodium tallowate and cocoate.

Pelletized soap 120 may also comprise about 0% to about 7.5% by weight of glycerin, about 2% to about 25% by weight of moisture, and about 0.8% of sodium chloride. In accordance with an exemplary embodiment of the invention, lathering and skin qualities of pelletized soap 120 may be achieved by having moisture level of the soap increased from about 8% to about 25% by weight.

In another exemplary embodiment, lathering and skin qualities of pelletized soap 120 may be achieved by having moisture level of the soap increased from about 18% to about 22% by weight. The preferred level of glycerin in the soap composition is from about 0.4% to about 2% by weight. The most preferred level of glycerin in the soap composition is from about 1% to about 2% by weight. Pelletized soap may be configured to include skin conditioning agent comprising about 0 to about 10% by weight. In another preferred example, the level of skin conditioning agent comprises about 0.05% to about 5% by weight. In a most preferred example, the level of skin conditioning agent comprises about 2.5% to about 3.5% by weight. To improve the lathering of the product, about 0% to about 15% by weight of a synthetic surfactant may be added. In a preferred example, about 1% to about 8% by weight may be added. In a most preferred example, about 3.5% to about 5.0% by weight may be added.

In accordance with another exemplary embodiment of the invention, a mild anionic synthetic foaming surfactant such as sodium laureth sulfate or disodium laureth sulfosuccinate may be used. A nonionic surfactant such as an alkyl glucoside or alkyl polyglucoside may also be used. Furthermore, an amphoteric lathering surfactant such as the higher alkyl betaines may be used, for example, such an amphoteric comprises cocoamidopropyl betaine. PEG-8 at a level of about 1% may be used, wherein the PEG-8 may serve as an emollient, emulsifier and surfactant. Soap composition may further incorporate antibacterial agents such as Triclosan or other well known antibacterials into the product.

Tulle pouch 130 may be made from any materials that function to retain pelletized soap 120 within tulle pouch 130. Moreover, tulle pouch 130 may include materials similarly used to manufacture mesh body 110. For example, tulle pouch 130 comprises a fine net of rayon or nylon or polypropylene. However, other combinations of synthetic materials now known or as yet unknown that function as noted above may likewise be used.

As noted above, tulle pouch 130 prevents pelletized soap 120 from falling out of pouf 100; however, in various embodiments it has also been discovered that by placing pelletized soap 120 within tulle pouch 130, the life of pelletized soap 120 is extended almost three times as long as comparable pieces of soap that are placed merely within mesh body 110, but not within an inner tulle pouch.

For instance, in an exemplary experiment, device 100, containing soap of the dimensions listed in Table 1, was tested using a Tergotometer Model 7243ES comprising six pots and six agitator shafts. The bath of the Tergotometer was heated to 95° F. and held there for the duration of the study. Each of the pots was filled three quarters full with 1,200 milliliters of water. Each pouf device 100 used in the test was submerged in the water of the pot prior to starting the washing machine agitation in each pot. The above washing action of the Tergotometer was conducted for about 12 minutes, devices 100 removed, and placed on hangers for five minutes to allow water to drain and then weigh each device. Devices 100 were placed back in the pots and further agitated for about 12 minutes. Following the period of agitation, devices 100 were again removed from the pots, drained for 5 minutes and weighed.

This cycle continued until all pelletized soap 120 within tulle pouch 130 of each device 100 was expended. Pelletized soap 120 comprises the following dimensions (inches):

TABLE 1
Soap Size (inches) (length × diameter)

¼ × ¼
¼ × ½
¼ × 2
⅝ × 2¼

Also, several solid pieces of water soluble fatty acid type soap (not pelletized) (weighing approximately 25 and 80 grams, respectively) were tested. The soap pellets placed in tulle pouch 130 weighed 25 grams per tulle pouch. The soap pieces not placed in tulle pouch 130 weighed 80 grams. Surprisingly, pouf device 100 having tulle pouch 130 that contained 25 grams of soap, displayed an average life of 91% of the life of the 80 gram soap in device 100 without use of tulle pouch 130 inner bag.

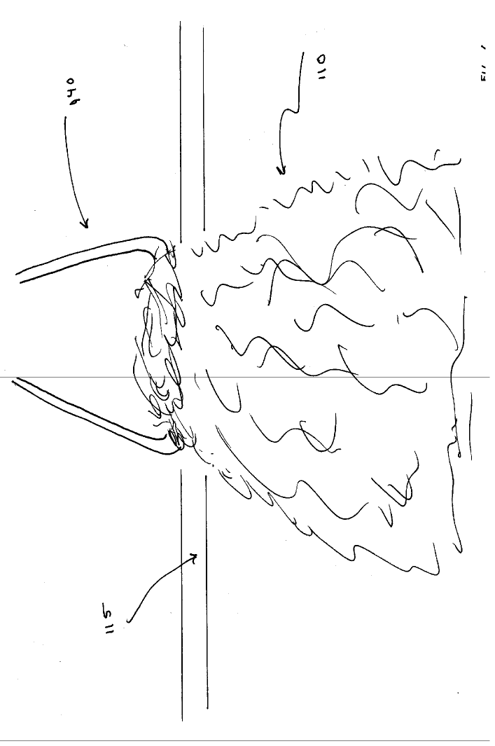

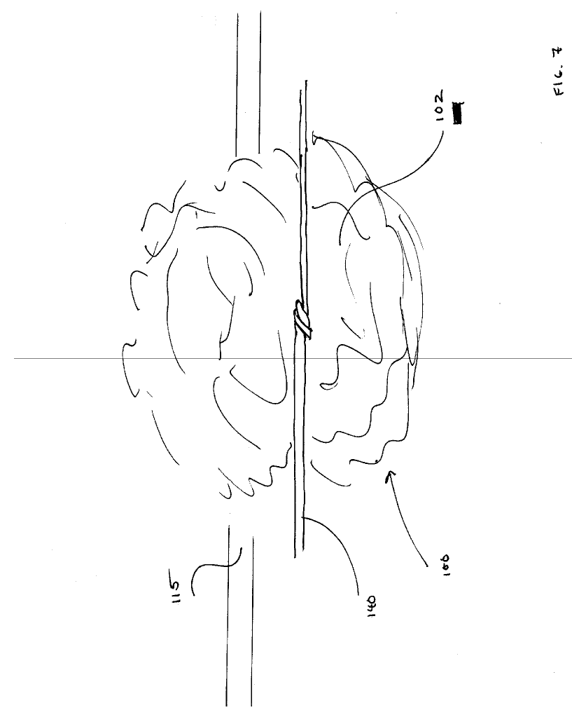

As briefly mentioned above, in accordance with an exemplary embodiment of the invention, and generally illustrated by FIGS. 6 and 7, device 100 comprises lanyard 140. Lanyard 140 comprises a rope type material, for example polyester. Lanyard 140 comprises a length of about 48 centimeters having a diameter of about 4 millimeters. Lanyard 140 is configured to maintain the position of tulle pouch 130 within mesh body 110, described in greater detail below. Lanyard 140 is also configured to secure the gathering of mesh body 110 into the desired shape, for example, an elongated body about horizontal plane 103 having generally flat upper and lower regions 102 and 104, as illustrated in FIG. 1. Additionally, lanyard 140 is further configured to effectuate handling of device 100 by a user, and also to facilitate storage of device 100 when not in use, by tying the ends in a loop and hanging from a support structure. It should be noted that lanyard 140 may comprise varying lengths and diameters of various materials, other than those described herein, and depending on the needs of the user.

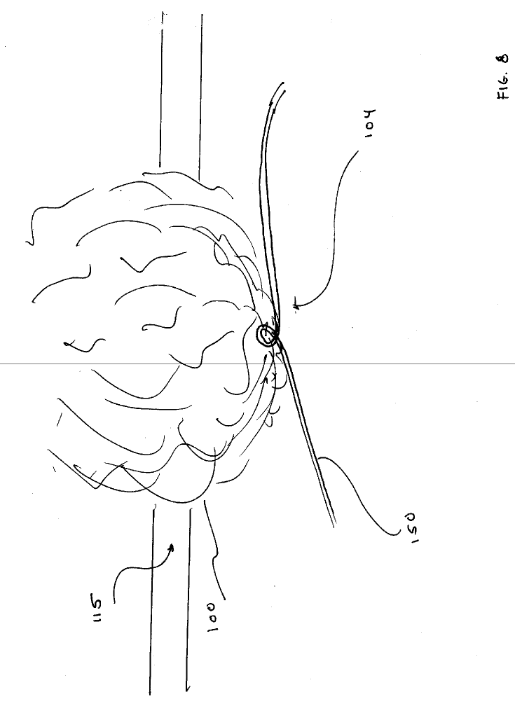

As also briefly mentioned above and in accordance with an exemplary embodiment of the invention, as generally illustrated by FIG. 8, device 100 comprises a secondary string 150. Secondary string 150 comprises a string type material, for example polyester. Secondary string 150 comprises a length of about 30 centimeters in length having a diameter of about 0.25 millimeters. Secondary string 150 is also configured to secure the gathering of mesh body 110 into the desired shape, for example an elongated body about horizontal plane 103 having generally flat upper and lower regions 102 and 104 as illustrated in FIG. 1. After securing mesh body 110 with secondary string 150, for example, by tying with a knot, any excess secondary string 150 is cut off and discarded. It should be noted that similar to lanyard 140, secondary string 150 may comprise varying lengths and diameters of various materials, other than those described herein, and depending on the needs of the user.

As illustrated in FIG. 3, to form the mesh cleansing device 100 as generally illustrated by FIG. 2, first, an elongated tubular piece of mesh that will form the mesh body 110, is cut to an appropriate length. Then both open ends are folded inside tubular piece 110 approximately 10-12 centimeters.

Next, as illustrated in FIG. 3, tubular piece 110 is placed over two substantially parallel rods 115, wherein rods 115 are separated by a distance that allows the placement of the internal tulle pouch 130. In one example, rods 115 are separated approximately 15 centimeters apart. As illustrated in FIG. 3, a tulle pouch 130 is positioned within the elongated tubular piece of mesh at approximately the mid-point.

Next, as illustrated in FIGS. 6 and 7, the elongated tubular piece of mesh material 110 on either side of tulle pouch 130 is gathered together to form a series of pleats. In one embodiment, there may be 0 to about 20 pleats, preferably about 2 to about 6 pleats, most preferably about 4 pleats. To secure the gathering of the elongated tubular piece of mesh material 110, and secure tulle pouch 130 to the interior of gathered mesh body 110, a cinch cord or lanyard 140 is tied around the series of pleats and tulle pouch 130 to band them together. The gathering secured by lanyard 140 is tightened so that pouf 100 is approximately 30 centimeters long.

Finally, as depicted in FIG. 8, secondary string 150 secures the gathering of the elongated tubular piece of mesh material on the side opposite the gathering secured by lanyard 140. Pouf device 100 is then separated from the rods and the final product is inspected.

Benefits, other advantages, and solutions to problems have been described herein with regard to specific embodiments. However, the benefits, advantages, solutions to problems, and any element(s) that may cause any benefit, advantage, or solution to occur or become more pronounced are not to be construed as critical, required, or essential features or elements of any or all the claims or the invention. The scope of the invention is accordingly to be limited by nothing other than the appended claims, in which reference to an element in the singular is not intended to mean “one and only one” unless explicitly so stated, but rather “one or more.” All structural, chemical, and functional equivalents to the elements of the above-described exemplary embodiments that are known to those of ordinary skill in the art are expressly incorporated herein by reference and are intended to be encompassed by the present claims.

What is claimed is:

1. A cleansing device comprising:
pelletized soap;
a tulle pouch configured to encase said soap; and
a pouf configured to encase tulle pouch.

2. The cleansing device of claim 1, wherein said tulle pouch has openings from about 60 to about 120 openings per square centimeter.

3. The cleansing device of claim 1, wherein said pouf has openings from about 1 to about 65 openings per square centimeter.

4. The cleansing device of claim 1, further comprising a tulle-pouf opening ratio defined as the ratio of the number of openings per square centimeter in the tulle pouch to the number of openings per square centimeter in the pouf, wherein said tulle-pouf opening ratio is about 50:1 to about 1:1.

5. The cleansing device of claim 1, further comprising a tulle-pouf opening ratio defined as the ratio of the number of openings per square centimeter in the tulle pouch to the number of openings per square centimeter in the pouf, wherein said tulle-pouf opening ratio is about 4:1 to about 1:1.

6. The cleansing device of claim 1, further comprising a tulle-pouf opening ratio defined as the ratio of the number of openings per square centimeter in the tulle pouch to the number of openings per square centimeter in the pouf, wherein said tulle-pouf opening ratio is about 3:1 to about 1:1.

7. The cleansing device of claim 1, wherein the average cross-sectional area of a soap pellet is about 0.2 to about 9 square centimeters.

8. The cleaning device of claim 1, wherein weight of pelletized soap is about 10 to about 90 grams.

9. The cleaning device of claim 1, wherein weight of pelletized soap is about 40 to about 60 grams.

10. The cleansing device of claim 1, wherein said pelletized soap composition comprises:
about 50 to about 100 percent by weight of soap pellets;
about 0 to about 7.5 percent glycerin;
about 0 to about 10 percent skin conditioning agent;
about 0 to about 15 percent surfactant; and
said composition having a moisture level of about 2 to about 25 percent by weight.

11. The cleansing device of claim 10, wherein said pelletized soap composition comprises about 90 to about 95 percent by weight of soap pellets.

12. The cleansing device of claim 10, wherein said pelletized soap composition comprises about 1 to about 2 percent glycerin.

13. The cleansing device of claim 10, wherein said pelletized soap composition comprises about 2.5 to about 3.5 percent skin conditioning agent.

14. The cleansing device of claim 10, wherein said pelletized soap composition comprises about 3.5 to about 5 percent surfactant.

15. The cleansing device of claim 10, wherein said composition having a moisture level of about 10 to about 22 percent by weight.

16. A pelletized soap composition comprising:
about 50 to about 100 percent by weight of soap pellets;
about 0 to about 7.5 percent glycerin;
about 0 to about 10 percent skin conditioning agent;
about 0 to about 15 percent surfactant; and
said composition having a moisture level of about 2 to about 25 percent by weight.

17. The pelletized soap composition of claim 16, wherein said pelletized soap composition comprises about 90 to about 95 percent by weight of soap pellets.

18. The pelletized soap composition of claim 16, wherein said pelletized soap composition comprises about 1 to about 2 percent glycerin.

19. The pelletized soap composition of claim 16, wherein said pelletized soap composition comprises about 2.5 to about 3.5 percent skin conditioning agent.

20. The pelletized soap composition of claim 16, wherein said pelletized soap composition comprises about 3.5 to about 5 percent surfactant.

21. The pelletized soap composition of claim 16, wherein moisture level is about 8 to about 25 percent by weight.

22. The pelletized soap composition of claim 16, wherein moisture level is about 10 to about 22 percent by weight.

23. A cleansing device comprising:
pelletized soap pieces;
a tulle pouch configured to encase said pieces;
a pouf configured to encase said tulle pouch comprising a tulle-pouf opening ratio defined as the ratio of the number of openings per square centimeter in the tulle pouch to the number of openings per square centimeter in the pouf, wherein said tulle-pouf opening ratio is about 50:1 to about 1:1; and
a lanyard configured to secure the gathering of said pouf and facilitate storage of said pouf.

24. The cleansing device of claim 23, wherein said tulle pouch has openings from about 60 to about 120 openings per square centimeter.

25. The cleansing device of claim 23, wherein said pouf has openings from about 1 to about 65 openings per square centimeter.

26. The cleaning device of claim 23, wherein weight of pelletized soap is about 10 to about 90 grams.

27. The cleansing device of claim 23, wherein average cross-sectional area of a soap pellet is about 0.2 to about 9 square centimeters.

28. The cleansing device of claim 23, wherein said pelletized soap composition comprises:
about 50 to about 100 percent by weight of soap pellets;
about 0 to about 7.5 percent glycerin;
about 0 to about 10 percent skin conditioning agent;
about 0 to about 15 percent surfactant; and
said composition having a moisture level of about 2 to about 25 percent by weight.

29. The cleansing device of claim 28, wherein said pelletized soap composition comprises about 90 to about 95 percent by weight of soap pellets.

30. The cleansing device of claim 28, wherein said pelletized soap composition comprises about 1 to about 2 percent glycerin.

31. The cleansing device of claim 28, wherein said pelletized soap composition comprises about 2.5 to about 3.5 percent skin conditioning agent.

32. The cleansing device of claim 28, wherein said pelletized soap composition comprises about 3.5 to about 5 percent surfactant.

33. The cleansing device of claim 28, wherein moisture level is about 8 to about 25 percent by weight.

34. The cleansing device of claim 28, wherein moisture level is about 10 to about 22 percent by weight.

35. A cleansing device comprising:
pelletized soap pieces, said pieces having an average cross-sectional area of about 0.35 square centimeters to about 3.5 square centimeters;
a tulle pouch configured to encase said pieces having openings from about 60 to about 120 opening per square centimeters;
a pouf having openings from about 1 to 65 openings per square centimeters configured to encase said tulle pouch;
a lanyard configured to secure the gathering of said pouf and facilitate storage of said pouf; and
a tulle-pouf opening ratio defined as the ratio of the number of openings per square centimeter in the tulle pouch to the number of openings per square centimeter in the pouf, wherein said tulle-pouf opening ratio is about 50:1 to about 1:1.

36. The cleaning device of claim 34, wherein weight of pelletized soap is about 10 to about 90 grams.

37. The cleaning device of claim 34, wherein weight of pelletized soap is about 40 to about 60 grams.

38. The cleansing device of claim 34, wherein average cross-sectional area of a soap pellet is about 0.2 to about 9 square centimeters.

39. The cleansing device of claim 34, wherein said pelletized soap composition comprises:
about 50 to about 100 percent by weight of soap pellets;
about 0 to about 7.5 percent glycerin;
about 0 to about 10 percent skin conditioning agent;
about 0 to about 15 percent surfactant; and
said composition having a moisture level of about 2 to about 25 percent by weight.

40. The cleansing device of claim 38, wherein said pelletized soap composition comprises about 90 to about 95 percent by weight of soap pellets.

41. The cleansing device of claim 38, wherein said pelletized soap composition comprises about 1 to about 2 percent glycerin.

42. The cleansing device of claim 38, wherein said pelletized soap composition comprises about 2.5 to about 3.5 percent skin conditioning agent.

43. The cleansing device of claim 38, wherein said pelletized soap composition comprises about 3.5 to about 5 percent surfactant.

44. The cleansing device of claim 38, wherein moisture level is about 8 to about 25 percent by weight.

45. The cleansing device of claim 38, wherein moisture level is about 10 to about 22 percent by weight.

46. A method for manufacturing a cleansing device comprising the steps of:
extending a tubular pouf mesh around at least two spaced, substantially parallel bars;
positioning a tulle pouch within substantially the midpoint of said extended tubular pouf mesh;
gathering a first portion of said tubular pouf mesh and said tulle pouch;
securing said first portion gathering with a first securing means;
gathering a second portion of said tubular pouf mesh; and
securing said second portion gathering with a second securing means.

47. The method of claim 45 further comprising the step of placing a pelletized soap within said tulle pouch.

48. The method of claim 45, wherein the steps of gathering a first portion and gathering a second portion further comprises the steps of gathering said first portion and second portion into at least about 1 to about 20 pleats.
